(19) United States
(12) Patent Application Publication
(10) Pub. No.: US 2016/0270903 A1
(43) Pub. Date: Sep. 22, 2016

(54) SYSTEM AND METHOD FOR SPINE LIGAMENT RECONSTRUCTION

(71) Applicant: Arthrex, Inc., Naples, FL (US)

(72) Inventors: Peter J. Dreyfuss, Naple, FL (US); Michael A. Gallizzi, Durham, NC (US)

(21) Appl. No.: 14/662,060

(22) Filed: Mar. 18, 2015

(51) Int. Cl. A61F 2/08; A61F 2/44

(52) U.S. Cl. CPC A61F 2/0811; A61F 2/4455; A61F 2002/0864; A61F 2002/0876; A61F 2210/0004

(57) ABSTRACT

A simple and flexible non-synthetic ligament which easily conforms to a patient's anatomy and can be used independently or in combination with an intervertebral graft, implant or prosthesis. A single length of allograft or autograft is provided, per each side of the vertebral column, to replace and recreate the spinal ligament spanning adjacent vertebrae. The single length allograft or autograft is secured to spine pedicles with fixation devices such as bone screws like interference screws and/or anchors such as SwiveLock® or PushLock® anchors.

### FIELD OF THE INVENTION

The present invention relates to the field of surgery and, more specifically, to apparatus and methods for treating spinal deformities and other spinal conditions.

### BACKGROUND OF THE INVENTION

Surgical treatment of the spine typically requires resection of a section of a spinal ligament to provide access to a diseased or damaged intervertebral disc and/or to permit introduction of a fusion implant, bone graft or intervertebral disc prosthesis for support of the vertebral bodies. The bone graft, fusion implant or intervertebral disc return stability to the spinal column in compression and flexing; however, due to removal of the spinal ligament, the biomechanical characteristics of extension and torsional stability lost by the removal of the ligament must be replaced.

Current techniques involve the use of metal bone plates which are secured to the vertebral bodies with screw locking mechanisms. Conventional bone plates are rigid, however, and they significantly inhibit spine mobility. The screw locking mechanisms utilized with such plates are also relatively complicated and provide minimal flexibility with respect to fastener positioning, for example.

There is a need for an improved spinal ligament that ensures the stability of spinal vertebrae level(s) after spinal surgeries where ligamentous structures are removed during exposure of the site. Also needed are methods of treating spinal deformities and other conditions through fusion or non-fusion procedures, and with autograft or allograft tissue, to recreate ligaments secured at the spine pedicles. Surgical systems and procedures adapted to replace pedicle screws and rods, as well as synthetic ligament-like materials that are unable to emulate natural motion and strength for proper long term results, are also needed.

### SUMMARY OF THE INVENTION

The present invention provides a simple and flexible non-synthetic ligament which easily conforms to a patient's anatomy and can be used independently or in combination with an intervertebral graft, implant or prosthesis. A single length of allograft or autograft is provided, per side, to replace and recreate the spinal ligament spanning adjacent vertebrae. The single length allograft or autograft is secured to the spine pedicles with fixation devices such as bone screws (interference screws), fasteners and/or anchors such as SwiveLock® or PushLock® anchors. The biomechanical supporting characteristics of the allograft or autograft tissue approximate the characteristics of the ligament (e.g., anterior spinal) which it replaces, thereby providing appropriate support to the spine while also permitting normal spine mobility.

The present invention also provides a method of continuous spine ligament reconstruction by providing a single length of allograft or autograft per each side and extending along, and between, at least two adjacent vertebrae. The method includes the steps of inter alias (i) providing a single continuous tissue of allograft or autograft per each side of the spinal column; and (ii) attaching the single continuous tissue of allograft or autograft to the spinal column, on each side, with fixation devices such as bone screws (interference screws) and/or anchors such as SwiveLock® or PushLock® anchors. Methods of reconstruction of ligamentous structures along adjacent vertebrae, as well as methods of supporting adjacent vertebrae with a single-piece allograft or autograft tissue, are also provided.

These and other features and advantages of the invention will be more apparent from the following detailed description that is provided in connection with the accompanying drawings and illustrated exemplary embodiments of the invention.

### BRIEF DESCRIPTION OF THE DRAWINGS

FIG. 1 illustrates an exemplary spinal ligament repair of the present invention.

FIG. 2 illustrates an exemplary tip of a swivel anchor employed in the spinal ligament repair of FIG. 1.

FIG. 3 illustrates an exemplary interference screw employed in the spinal ligament repair of FIG. 1.

### DETAILED DESCRIPTION OF THE PREFERRED EMBODIMENTS

In the following detailed description, reference is made to various specific embodiments in which the invention may be practiced. These embodiments are described with sufficient detail to enable those skilled in the art to practice the invention, and it is to be understood that other embodiments may be employed, and that structural changes may be made without departing from the scope of the present invention.

The present invention provides a simple and flexible non-synthetic ligament which easily conforms to a patient's anatomy and can be used independently or in combination with an intervertebral graft, implant or prosthesis. A single length of allograft or autograft is provided, per side, to replace and recreate the spinal ligament spanning adjacent vertebrae, for example, the anterior longitudinal ligament and/or the posterior longitudinal ligament (i.e., ligaments that stabilize the spine). The single length allograft or autograft is secured to the vertebrae (for example, to the spine pedicles) with fixation devices such as bone screws (interference screws) and/or anchors such as SwiveLock® or PushLock® anchors. The biomechanical supporting characteristics of the allograft or autograft tissue approximate the characteristics of the ligament (e.g., supraspinous, interspinous, and yellow spinal ligaments) which it replaces, thereby providing appropriate support to the spine in extension while also permitting normal spine mobility.

The present invention also provides a method of continuous ligament reconstruction by providing a single length of allograft or autograft per each side of the vertebral column and extending along, and between, at least two adjacent vertebrae. The method includes the steps of inter alia: (i) providing a single continuous tissue of allograft or autograft per each side of the spinal column; and (ii) attaching the single continuous tissue of allograft or autograft to the spinal column, on each side, with fixation devices such as bone screws (interference screws) and/or anchors such as SwiveLock® or PushLock® anchors.

As detailed below, the autograft or allograft tissue has particular application in replacing the supportive function of a spinal ligament, for example, the supraspinous, interspinous, yellow or posterior longitudinal ligament, which may have been fully or partially resected during a spinal procedure. The autograft or allograft tissue is advantageously dimensioned to be positioned to span adjacent vertebrae to restore the natural biomechanics, e.g., tensional support and range of motion, of the removed ligament segment. The autograft or allograft tissue may be used with a bone graft, fusion implant, or artificial disc to complement the compressive load characteristics of the implant with its tensional supporting capabilities during healing.

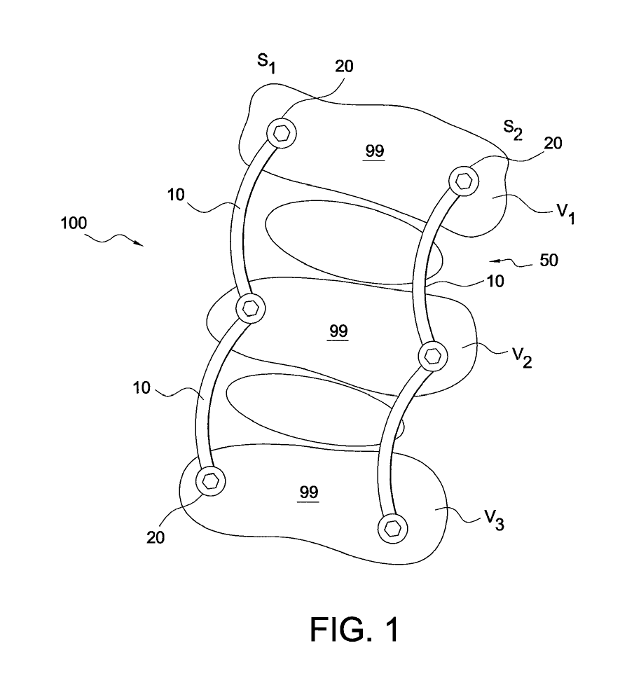

Referring now to the drawings, where like elements are designated by like reference numerals, FIG. 1 illustrates an exemplary spinal ligament repair 100 with system 50 of the present invention. System 50 includes at least one length 10 of allograft or autograft tissue that is provided, per each side of vertebrae V1-V3, to replace and recreate the spinal ligament spanning adjacent vertebrae, for example, the anterior longitudinal ligament and/or the posterior longitudinal ligament. System 50 also includes one or more fixation devices 20.

FIG. 1 illustrates for simplicity only three adjacent vertebrae V1-V3 with pedicles 99; however, the invention contemplates allograft or autograft tissue spanning any number of vertebrae. FIG. 1 also shows two lengths of tissue 10 (two allograft or autograft tissue 10) extending on each side S1, S2 of the vertebral column, i.e., on each side or margin S1, S2 of the spine pedicles of the vertebrae V1-V3.

Allograft or autograft tissue 10 is secured to the bone (vertebra) by employing fixation devices 20 which may be in the form of screws 20 such as interference screws, bone fasteners, non-threaded or threaded devices, expandable bolts, or anchors such as suture anchors or bone anchors.

According to an exemplary-only embodiment, the fixation devices 20 are threaded interference screws that allow secure attachment of allograft or autograft tissue 10 to bone holes formed within the pedicles of each one of vertebrae V1-V3. A plurality of interference screws 20 may be employed depending on the number of vertebrae to be spanned. The number of the interference screws is similar to that of the vertebrae. An exemplary interference screw 20 is shown in FIG. 3.

In another exemplary-only embodiment, the fixation devices 20 are knotless fixation devices such as swivel anchors, screw-in anchors or push-in suture anchors, or combinations thereof (such as an Arthrex SwiveLock® anchor, disclosed in U.S. Patent Application Publication No. 2008/0004659 or a PushLock® anchor, disclosed in U.S. Pat. No. 7,329,272).

The fixation devices may include one or more swivel anchors provided with a tip and with a shaft and an eyelet attached to the shaft, and a separate cannulated fixation device, wherein the tip with the eyelet is rotatable relative to the cannulated fixation device and is configured to swivel relative to the cannulated fixation device, the eyelet being optionally pre-loaded with a flexible strand that attaches to tissue 10. To anchor the swivel anchor into a socket formed within a vertebra such as vertebrae V1-V3, the cannulated fixation device is rotated over the shaft so that the cannulated fixation device advances over the shaft of the tip of the swivel anchor and engages and fully seats the tip and the eyelet within the vertebral socket.

The eyelet may be a closed aperture or closed eyelet that is pre-loaded with a flexible strand (which in turn attaches tissue 10 to the vertebrae and to the anchor). The flexible strand may form a loop around tissue 10 or, alternatively, the flexible strand may pass through the tissue 10.

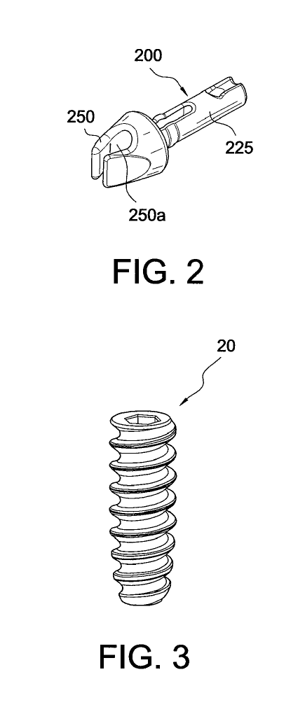

Alternatively, the eyelet may have a forked, open configuration to directly engage and secure the tissue 10 at the bottom of the hole or socket formed within each vertebra. An exemplary eyelet 200 is shown in FIG. 2. The eyelet 200 has a forked tip 250 provided with opening 250a. The forked configuration allows the tissue 10 to be received within opening 250a of the forked tip and secured within the vertebral hole or socket with the cannulated fixation device 20 (FIG. 3). To secure/anchor the swivel anchor into a socket formed within a vertebra such as vertebrae V1-V3, the cannulated fixation device 20 is rotated/advanced over the shaft 225 so that the cannulated fixation device 20 advances over the shaft 225 of the tip 200 of the swivel anchor and engages and fully seats the tip 200 and the eyelet 250 with tissue 10 secured/captured within opening 250a.

The knotless fixation devices 20 may be also knotless, adjustable, self-locking anchors which may be provided with a flexible strand (suture or similar materials) having a flexible closed continuous loop (suture loop) having an adjustable perimeter or length. The flexible strand may also include a splice which may be located within the anchor body (extending between the proximal end and the distal end) or outside the anchor body. The knotless fixation devices 20 may further include a shuttling device in the form of a suture passing instrument or a wire loop, to form the splice and the knotless, adjustable closed loop.

In yet another exemplary embodiment, the fixation devices 20 may include a SutureTak® anchor which is a knotless suture anchor with a splice-forming mechanism as set forth in U.S. Patent Publication No. 2013/0345750, entitled “Tensionable Knotless Labral Anchor and Methods of Tissue Repair,” U.S. Patent Publication No. 2013/0096611, entitled “Tensionable Knotless Anchors with Splice and Methods of Tissue Repair” and U.S. Patent Publication No. 2013/0165972, entitled “Tensionable Knotless Anchor Systems and Methods of Tissue Repair,” the disclosures of all of which are incorporated by reference in their entirety herein.

The knotless SutureTak® anchors may be modified to accommodate a larger suture to hold a larger allograft or autograft tissue 10. The suture could be interconnected to the adjacent anchors provided within bodies of adjacent vertebrae. Details of the formation of an exemplary knotless suture anchor employed in the embodiments of the present invention and with a splice-forming mechanism are set forth in U.S. Patent Publication No. 2013/0345750 entitled “Tensionable Knotless Labral Anchor and Methods of Tissue Repair,” U.S. Patent Publication No. 2013/0096611 entitled “Tensionable Knotless Anchors with Splice and Methods of Tissue Repair,” and U.S. Patent Publication No. 2013/0165972 entitled “Tensionable Knotless Anchor Systems and Methods of Tissue Repair,” the disclosures of all of which are incorporated by reference in their entirety herein.

In an exemplary embodiment, the knotless fixation device 20 may be a self-cinching knotless adjustable construct which is pre-loaded with a tensionable construct formed of a flexible strand (suture) attached to a shuttling device (a shuttle/pull device or suture passing device for example, a FiberLink™ or a nitinol loop). The shuttling device is configured to be pulled out of the body of the fixation device (anchor) to allow the flexible strand to pass through itself and form a splice within the body of the fixation device (or outside of the body of the fixation device). The body of the fixation device is cannulated and the tensionable construct extends through the body of the fixation device. The body is provided with a proximal end that receives a tip of a driver and a distal end that is configured to house a knot of the flexible strand, so that the flexible strand has only one free end. The flexible strand may loop around the autograft or allograft tissue 10.

The tensionable knotless anchors may be provided with the splice-forming mechanism, or may be provided pre-loaded with the splice, i.e., with no shuttle/pull device attached to the suture (no shuttle/pull device necessary). The tensionable knotless anchors may be used to achieve simple repairs, such as a simple single-stitch repair on each vertebra while securing the tissue 10 to each vertebra.

In use, and in connection with an anterior spinal procedure, the native anterior ligament (or at least a portion thereof) is removed to permit access to a diseased or damaged disc section. A partial or full discectomy may be performed followed by insertion of a bone graft, fusion implant or an intervertebral prosthesis. A method of continuous ligament reconstruction by providing a single length of allograft or autograft per side of the vertebral column and extending along, and between, at least two adjacent vertebrae includes inter alia the steps of: (i) providing a single continuous tissue 10 of allograft or autograft per each side S1, S2 of the spinal column; and (ii) securing the single continuous tissue 10 of allograft or autograft to bone (vertebra), at each side S1, S2, with fixation devices 20 such as bone screws (interference screws) and/or anchors such as SwiveLock® or PushLock® anchors. The step of securing the tissue may be conducted with or without the use of flexible material (for example, suture, suture chain, suture tape, etc.).

A method of spine ligament reconstruction with system 50 of the present invention includes inter alia the steps of: (i) providing a first and a second single continuous tissue 10 of allograft or autograft on a first side S1 and on a second side S2 of the spinal column, respectively, the first and the second single continuous tissue 10 of allograft or autograft extending along the first side S1 and the second side S2, respectively, and between at least two adjacent vertebrae V1-V3; (ii) securing the first and the second single continuous tissue of allograft or autograft, on/at the first side S1 and on/at the second side S2, respectively, with fixation devices 20 such as bone screws (interference screws) and/or anchors such as SwiveLock® or PushLock® or SutureTak® anchors.

The present invention also provides methods of spine ligament reconstructions which do not require tying of knots and allow adjustment of both the tension of the suture and the location of the tissue with respect to the bone/vertebra.

An exemplary method of spine ligament reconstruction according to the present invention, comprises the steps of: (i) removing a portion of a native spinal ligament; (ii) replacing the portion of the native spinal ligament with a continuous length of autograft or allograft tissue 10; and (iii) securing the continuous length of autograft or allograft tissue 10 to adjacent vertebrae V1-V3, with fixation devices 20, to reproduce the portion of the native spinal ligament. The fixation devices 20 may be selected from the group consisting of interference screws, bone anchors, swivel anchors and expandable devices. In one embodiment, at least one of the fixation devices 20 is a swivel anchor provided with a tip 200 and with a shaft 225 and an eyelet 250 having a forked configuration attached to the shaft 225, and a separate cannulated fixation device 20, wherein the tip 200 with the eyelet 250 is rotatable relative to the cannulated fixation device 20 and is configured to swivel relative to the cannulated fixation device 20.

The method further comprises the steps of: (iv) capturing the continuous length of autograft or allograft tissue 10 with the forked tip 200 of the swivel anchor so that the tissue 10 rests within opening 250a of the forked eyelet 250; (v) inserting the captured continuous length of autograft or allograft tissue 10 with the forked tip 200 within a hole or socket formed within a spine pedicle of a vertebra V; and (vi) securing the captured continuous length of autograft or allograft tissue 10 within the hole or socket by advancing the cannulated fixation device 20 over the tip 200 of the swivel anchor, so that the cannulated fixation device 20 engages and fully seats the tip 200 and the eyelet 250 attached to the at least one continuous length of autograft or allograft tissue 10 within the hole or socket.

If one or more flexible strands are employed, the flexible strands or materials may be in the form of any suture, tape or chain, or combinations thereof. In an exemplary embodiment only, the flexible material is in the form of a tape which may include a wide selection of synthetic compositions such as ultrahigh molecular weight polyethylene (UHMWPE), or the FiberWire® suture described in U.S. Pat. No. 6,716,234, or the FiberTape® suture tape described in U.S. Pat. No. 7,892,256, the disclosures of both of which are incorporated by reference in their entirety herewith. FiberTape® is a suture tape (flat braid) with an ultra-high strength, about 2 mm wide, and with a structure similar to that of the FiberWire® suture. The tape may provide broad compression and increased tissue cut-through resistance.

Although the present invention has been described in connection with preferred embodiments, many modifications and variations will become apparent to those skilled in the art. While preferred embodiments of the invention have been described and illustrated above, it should be understood that these are exemplary of the invention and are not to be considered as limiting. Accordingly, it is not intended that the present invention be limited to the illustrated embodiments, but only by the appended claims.

What is claimed as new and desired to be protected by Letters Patent of the United States is:

1. A system for correcting or treating a spinal deformity, comprising:
at least one continuous length of autograft or allograft tissue for recreating ligaments secured at spine pedicles; and
fixation devices for attaching the at least one continuous length of autograft or allograft tissue to the spine pedicles.

2. The system of claim 1, wherein the fixation devices are threaded or un-threaded devices.

3. The system of claim 1, wherein the fixation devices are screws or anchors.

4. The system of claim 3, wherein the fixation devices are interference screws.

5. The system of claim 3, wherein at least one of the fixation devices is a swivel anchor provided with a tip and with a shaft and an eyelet attached to the shaft, and a separate cannulated fixation device, wherein the tip with the eyelet is rotatable relative to the cannulated fixation device and is configured to swivel relative to the cannulated fixation device.

6. The system of claim 5, wherein the eyelet is pre-loaded with a flexible strand that forms a loop around the at least one continuous length of autograft or allograft tissue.

7. The system of claim 5, wherein the eyelet has an open forked configuration to allow the at least one continuous length of autograft or allograft tissue to be captured therein and secured within a hole or socket in the spine pedicle.

8. The system of claim 5, wherein, when the swivel anchor is anchored into a socket of a spine pedicle of a vertebra, the cannulated fixation device is configured to be advanced over the shaft so that the cannulated fixation device advances over the shaft of the tip of the swivel anchor and engages and fully seats the tip and the eyelet attached to the at least one continuous length of autograft or allograft tissue.

9. The system of claim 1, comprising two continuous lengths of autograft or allograft tissue expanding at least two adjacent vertebrae, and a plurality of swivel anchors.

10. The system of claim 1, comprising two continuous lengths of autograft or allograft tissue expanding at least two adjacent vertebrae, and a plurality of interference screws.

11. A method of spine ligament reconstruction, comprising the steps of:
removing a portion of a native spinal ligament;
replacing the portion of the native spinal ligament with a continuous length of autograft or allograft tissue; and
securing the continuous length of autograft or allograft tissue to adjacent vertebrae, with fixation devices, to reproduce the portion of the native spinal ligament.

12. The method of claim 11, wherein the fixation devices are selected from the group consisting of interference screws, bone anchors, swivel anchors and expandable devices.

13. The method of claim 11, wherein at least one of the fixation devices is a swivel anchor provided with a tip and with a shaft and an eyelet having a forked configuration attached to the shaft, and a separate cannulated fixation device, wherein the tip with the eyelet is rotatable relative to the cannulated fixation device and is configured to swivel relative to the cannulated fixation device, and wherein the method further comprises the steps of:
capturing the continuous length of autograft or allograft tissue with the forked tip of the swivel anchor;
inserting the captured continuous length of autograft or allograft tissue with the forked tip within a hole or socket formed within a spine pedicle of a vertebra; and
securing the captured continuous length of autograft or allograft tissue within the hole or socket by advancing the cannulated fixation device over the tip of the swivel anchor so that the cannulated fixation device engages and fully seats the tip and the eyelet attached to the at least one continuous length of autograft or allograft tissue within the hole or socket.

14. The method of claim 11, wherein the native spinal ligament is the posterior ligament.
